(19) United States
(12) Patent Application Publication
(10) Pub. No.: US 2025/0250531 A1
(43) Pub. Date: Aug. 7, 2025

(54) METHOD FOR ISOLATION AND CHARACTERIZATION OF NUCLEUS PULPOSUS PROGENITOR CELLS AND FOR IMPROVING LOWER BACK PAIN USING NUCLEUS PULPOSUS PROGENITOR CELLS

(71) Applicant: ASTEROGENE Biomedical Co., Ltd., New Taipei City (TW)

(72) Inventors: WEI-HUNG CHEN, New Taipei City (TW); SHIH-CHEN TSAI, New Taipei City (TW)

(73) Assignee: ASTEROGENE Biomedical Co., Ltd., New Taipei City (TW)

(21) Appl. No.: 18/641,574

(22) Filed: Apr. 22, 2024

(30) Foreign Application Priority Data
Feb. 6, 2024 (TW) 113104672

(51) Int. Cl. C12M 1/00; A61K 35/12; A61P 25/04; C12Q 1/6881

(52) U.S. Cl. CPC C12M 47/04; A61K 35/12; A61P 25/04; C12Q 1/6881

(57) ABSTRACT

The invention primarily relates to a method for isolating and characterizing nucleus pulposus progenitor cells. The method initially involves cutting nucleus pulposus tissue into multiple tissue blocks and then enzymatically digesting these blocks. After digestion, these tissue blocks are cultured in a culture dish. When cells form clusters at the bottom of the dish, specific embryonic stem cell genes (such as Nanog, Oct-4, SOX2) are used to select nucleus pulposus progenitor cells from the clusters. Furthermore, this method employs specific proteins and genetic markers to select nucleus pulposus progenitor cells with mobility and/or mesenchymal stem cell characteristics, as well as to further select cells with anti-inflammatory abilities. Finally, the invention discloses a use of the nucleus pulposus progenitor cells obtained by the method in the manufacture of a pharmaceutical composition for the treatment of lower back pain, offering a new strategy for the treatment of lower back pain.

### BACKGROUND OF THE INVENTION

### 1. Field of the Invention

The present invention relates to the technical field of cell isolation and characterization methods, in particular a method for isolating and characterizing nucleus pulposus progenitor cells with migratory and anti-inflammatory properties from the nucleus pulposus tissue of human intervertebral discs.

### 2. Description of the Prior Art

The intervertebral disc is a soft tissue structure located between the vertebrae of the human spine, primarily composed of the outer annulus fibrosus and the inner nucleus pulposus tissue. These intervertebral discs play a crucial role in cushioning and supporting the vertebrae, thereby helping to reduce friction and impact between them. Degeneration of these intervertebral discs often leads to lower back pain in adults, affecting their daily life and work efficiency.

In traditional treatment methods, the initial stages of intervertebral disc degeneration are primarily treated with medications, such as nonsteroidal anti-inflammatory drugs (NSAIDs) and steroids, to alleviate pain and inflammation. However, long-term use of these medications can lead to gastrointestinal issues and adversely affect cellular function. For severe cases of intervertebral disc degeneration, more invasive treatments may be necessary, such as intervertebral disc replacement surgery. However, this approach carries risks and is costly.

When the intervertebral disc is damaged or degenerated, the nucleus pulposus tissue may deform or protrude, potentially leading to nerve compression. This can cause symptoms such as pain, numbness, or muscle weakness. Current treatment methods often fail to directly address the fundamental issue of the intervertebral disc, namely the degeneration and damage of the nucleus pulposus tissue.

Given the limitations of existing treatments for intervertebral disc degeneration, the present invention proposes an innovative method aimed at isolating and characterizing nucleus pulposus progenitor cells, which possess migratory and anti-inflammatory properties, from the nucleus pulposus tissue of the patient's intervertebral disc. The method of the present invention involves isolating nucleus pulposus progenitor cells from the patient's nucleus pulposus tissue during the early stages of intervertebral disc degeneration. From these isolated cells, nucleus pulposus progenitor cells with stem cell characteristics, as well as migratory and anti-inflammatory properties, are screened and identified. These cells are then cultured and amplified in vitro, so they can be re-implanted into the patient when needed, thereby promoting the regeneration and repair of intervertebral disc tissue. The innovation of this method lies in its ability to directly treat intervertebral disc degeneration and significantly improve the patient's quality of life.

### SUMMARY OF THE INVENTION

The main object of the present invention is to provide a method for isolating and characterizing nucleus pulposus progenitor cells, comprising:
a) providing a nucleus pulposus tissue, and cutting the nucleus pulposus tissue into a plurality of tissue blocks;
b) hydrolyzing the tissue blocks with an enzyme;
c) removing the enzyme and culturing the tissue blocks in a culture dish; and
d) when a plurality of cell populations emerge from the tissue blocks and form a plurality of clusters at the bottom of the culture dish, screening for the nucleus pulposus progenitor cells within each of the cell populations in each cluster, using an embryonic stem cell gene, wherein the embryonic stem cell gene is selected from at least one of the group consisting of Nanog, Oct-4, and SOX2;
wherein the enzyme used for hydrolyzing the tissue blocks is collagenase, trypsin, or a combination thereof.

In the method for isolating and characterizing nucleus pulposus progenitor cells, the nucleus pulposus progenitor cells with migratory property can be further screened using at least one protein selected from the group consisting of N-Cadherin, Vimentin, β-Catenin, and Snail protein.

In the method for isolating and characterizing nucleus pulposus progenitor cells, the nucleus pulposus progenitor cells with mesenchymal stem cell characteristics can be further screened using at least one gene selected from the group consisting of STRO-1, C-KIT, β-catenin, Jagged, and Delta4.

In the method for isolating and characterizing nucleus pulposus progenitor cells, the nucleus pulposus progenitor cells with mesenchymal stem cell characteristics can be further screened using at least one stem cell surface antigen selected from the group consisting of CD34, CD44, CD73, CD90, CD105, and CD133.

Furthermore, in the method for isolating and characterizing nucleus pulposus progenitor cells, the nucleus pulposus progenitor cells with a differentiation capability can be further screened using the differentiation capability selected from the group consisting of chondrogenesis, osteogenesis, and adipogenesis.

Additionally, in the method for isolating and characterizing nucleus pulposus progenitor cells, the nucleus pulposus progenitor cells with an anti-inflammatory capability can be further screened using at least one inflammation-related gene selected from the group consisting of IL-1β, COX-2, and MMP3.

Meanwhile, the second object of the present invention is to provide a method for improving lower back pain, comprising administering an effective amount of a pharmaceutical composition containing the nucleus pulposus progenitor cells obtained by the above-mentioned method for isolating and characterizing nucleus pulposus progenitor cells to a subject suffering from lower back pain.

In the method for improving lower back pain, the nucleus pulposus progenitor cells with migratory properties can be further screened using at least one protein selected from the group consisting of N-Cadherin, Vimentin, β-Catenin, and Snail protein, and/or the nucleus pulposus progenitor cells with mesenchymal stem cell characteristics can be further screened using at least one stem cell surface antigen selected from the group consisting of CD34, CD44, CD73, CD90, CD105, and CD133.

In the method for improving lower back pain, the nucleus pulposus progenitor cells with a differentiation capability can be further screened using the differentiation capability selected from the group consisting of chondrogenesis, osteogenesis, and adipogenesis; and/or the nucleus pulposus progenitor cells with an anti-inflammatory capability can be further screened using at least one inflammation-related gene selected from the group consisting of IL-1β, COX-2, and MMP3.

The nucleus pulposus progenitor cells obtained from a patient's nucleus pulposus tissue can be expanded in culture and then re-transplanted into the degenerated site of the patient's intervertebral disc through the pharmaceutical composition. This allows the nucleus pulposus progenitor cells to grow in the affected area of the degenerated intervertebral disc, achieving therapeutic purposes through the regeneration of nucleus pulposus tissue, and minimizing the possibility of immune rejection in patents who receive the transplantation.

### BRIEF DESCRIPTION OF THE DRAWINGS

FIG. 1 shows the state of cell populations emerging from tissue blocks and growing at the bottom of the culture dish on days 1 and 4 of culture;

FIGS. 2A-2D show the results of characterizing nucleus pulposus progenitor cells using embryonic stem cell genes Nanog, Oct-4, and SOX2;

FIGS. 3A-3B show the results of the tests for migratory properties conducted on the screened nucleus pulposus progenitor cells;

FIGS. 4A-4D show the characterization of cell populations selected based on mesenchymal stem cell (MSC) characteristics;

FIGS. 5A-5D show the results of tests for anti-inflammatory capabilities conducted on the selected cell populations; and

FIG. 6 is a flowchart of the method for isolating and characterizing nucleus pulposus progenitor cells according to the present invention.

### DETAILED DESCRIPTION OF THE PREFERRED EMBODIMENTS

Unless defined otherwise, all terms, including technical and scientific terms, used herein have the same meaning as commonly understood by a person of ordinary skill in the art to which the present inventive concepts pertain. The present invention will be further elucidated through the following exemplary embodiments; however, the present invention is not limited by these exemplary embodiments. Unless otherwise specified, the materials used in the present invention are commercially available, and the examples provided are merely illustrative of commercially available options.

### Embodiment 1: Processing Sample of Nucleus Pulposus Tissue

1. A sample of nucleus pulposus tissue from a patient with disc degeneration or disc herniation is aseptically removed during surgery in a hospital operating room. The sample of nucleus pulposus tissue is then placed in a sterile closed container containing an antibiotic composition combined with physiological saline solution and sent to a sterile laminar flow cabinet in a Good Tissue Practice (GTP) laboratory for further processing.
2. Record the weight of the nucleus pulposus tissue sample and the basic information of the patient from whom the sample was obtained.
3. Place the nucleus pulposus tissue sample in a 10 cm petri dish containing 2 mL of medium, and then cut the nucleus pulposus tissue sample into small tissue blocks with an average size of less than 1 mm³. Approximately 5 g of tissue blocks are used for one 10 cm petri dish, and if there are more tissue blocks, they are divided into multiple petri dishes accordingly. The medium comprises the following components: DMEM (Dulbecco's Modified Eagle Medium), human platelet lysate, and antibiotics.
4. Prepare a collagenase solution by mixing 7 mL of medium with 1 mL of type I collagenase, resulting in a 7:1 volume ratio of medium to type I collagenase.
5. Add the prepared collagenase solution to the petri dishes containing the tissue blocks. Add 8 ml of the collagenase solution to each petri dish and incubate for 4 to 24 hours in a cell culture incubator at 37° C. with 5% CO₂.
6. After 4 to 24 hours of incubation, rinse the tissue blocks from each petri dish with 10 mL of DPBS (Dulbecco's phosphate-buffered saline) to remove the collagenase solution.
7. Transfer the tissue blocks and culture them in a 10 cm petri dish containing 10 mL of medium for 1˜7 days.
8. When a plurality of cell populations emerge from the tissue blocks and form a plurality of clusters at the bottom of the petri dish, collect each cell population in each cluster separately and culture them separately in different culture dishes.
9. Then, screen for nucleus pulposus progenitor cells from each collected and cultured cell population in each cluster using an embryonic stem cell gene.

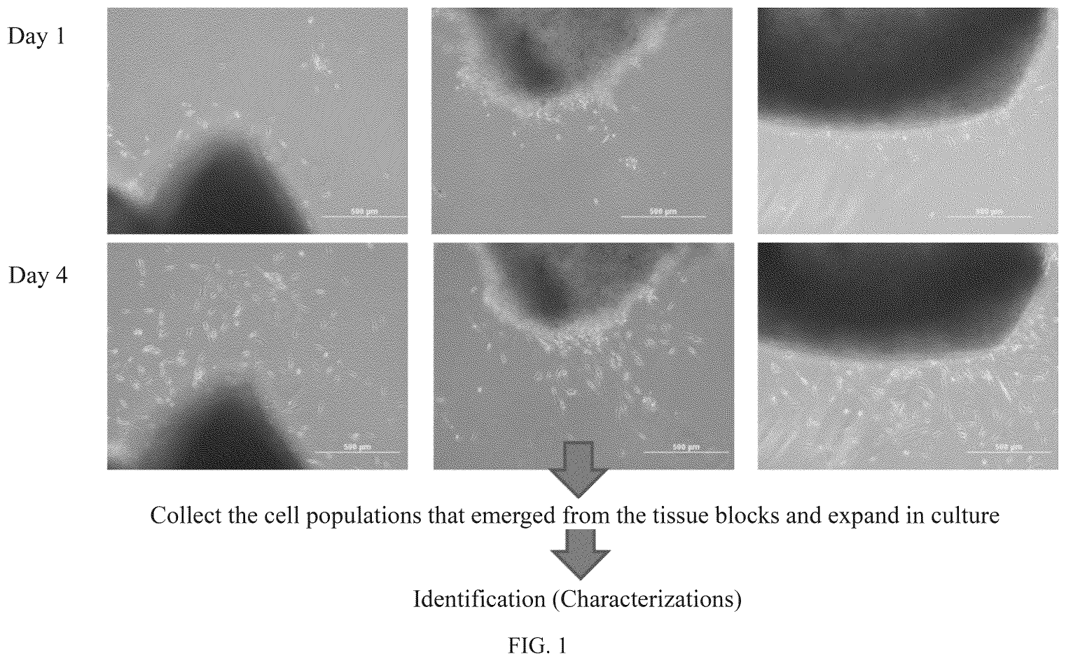

FIG. 1 shows the condition of the tissue blocks on day 1 and day 4 of culture. It is clearly visible that after 4 days of culture, the number of cell populations that have emerged from the tissue blocks and grown at the bottom of the culture dish has significantly increased, compared to the smaller number of cell populations on day 1. Subsequently, these emerged cell populations growing in different clusters at the bottom of the culture dish are collected and transferred to different culture dishes for further expansion culture. Finally, these cells undergo identification (characterization), such as screening for nucleus pulposus progenitor cells using embryonic stem cell genes Nanog, Oct-4, and SOX2.

### Embodiment 2: Identification of Nucleus Pulposus Progenitor Cells Using Embryonic Stem Cell Genes

In this embodiment, after the different cell populations grown in different sections at the bottom of the petri dish are collected and cultured, nucleus pulposus progenitor cells are further screened from these cell populations using the embryonic stem cell genes Nanog, Oct-4, and SOX2, with mesenchymal stem cells (MSC) and parental nucleus pulposus cell population (Parental) as control groups to compare the expression ratio of embryonic stem cell genes in these cell populations. A selection threshold of greater than 1.5 in gene expression ratio is set, meaning cell populations with an embryonic stem cell gene expression ratio greater than 1.5 meet the selection criteria. Moreover, cell populations that meet the selection criteria for all three genes (Nanog, Oct-4, and SOX2) are selected for further analysis.

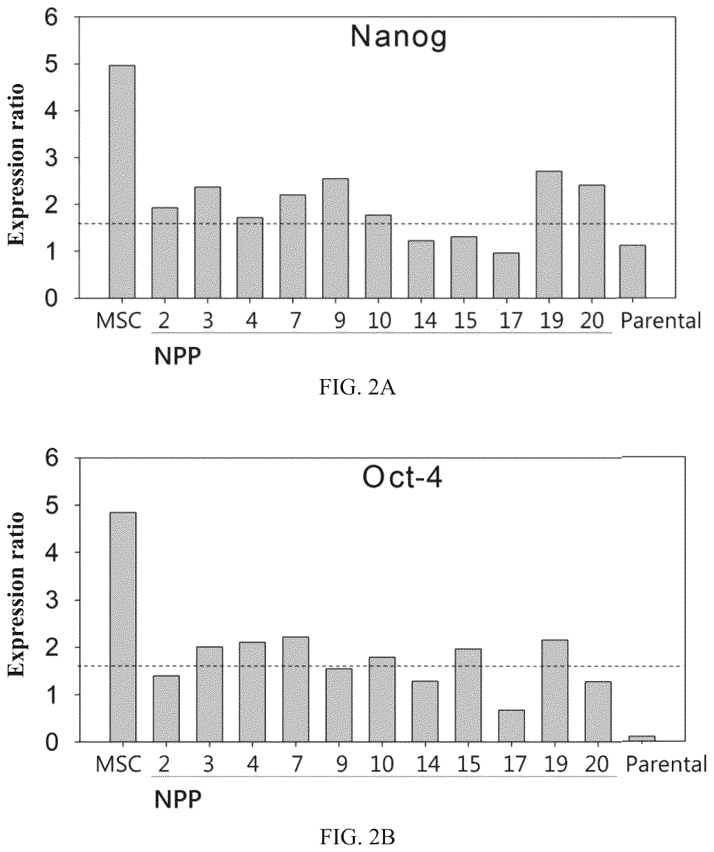

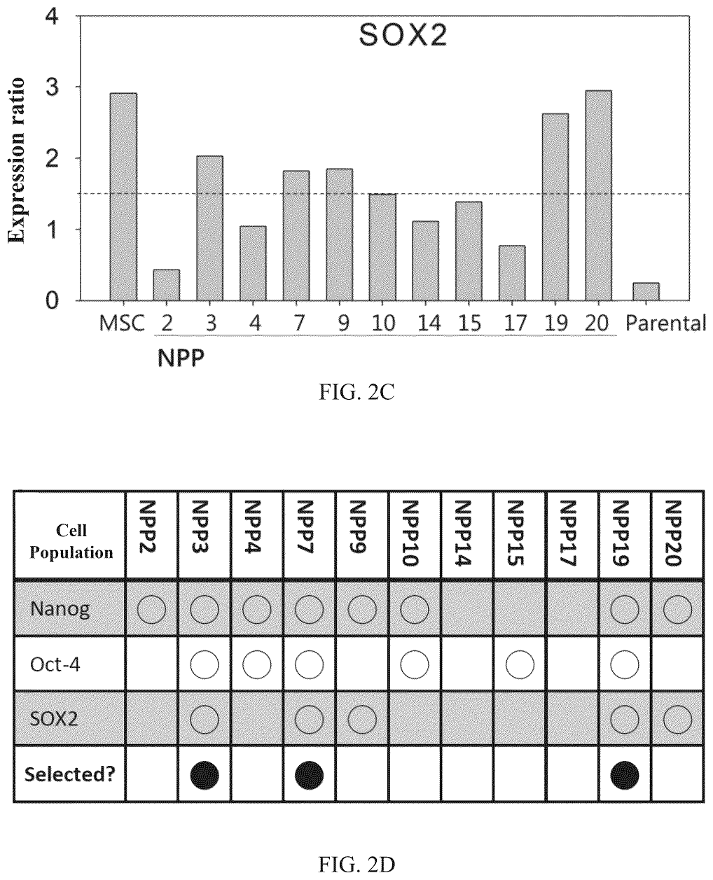

FIG. 2A shows the analysis results of different cell populations (NPP2, 3, 4, 7, 9, 10, 14, 15, 17, 19, 20) using the Nanog gene, with cell populations NPP2, 3, 4, 7, 9, 10, 19, 20 meeting the selection threshold of having an expression ratio greater than 1.5.

FIG. 2B shows the analysis results of different cell populations (NPP2, 3, 4, 7, 9, 10, 14, 15, 17, 19, 20) using the Oct-4 gene, with cell populations NPP3, 4, 7, 10, 15, 19 meeting the selection threshold of having an expression ratio greater than 1.5.

FIG. 2C shows the analysis results of different cell populations (NPP2, 3, 4, 7, 9, 10, 14, 15, 17, 19, 20) using the SOX2 gene, with cell populations NPP3, 7, 9, 19, 20 meeting the selection threshold of having an expression ratio greater than 1.5.

The chart of FIG. 2D shows the preliminary screening results ofnucleus pulposus progenitor cells from the aforementioned cell populations using the embryonic stem cell genes Nanog, Oct-4, and SOX2. Only cell populations that meet the selection threshold for all three genes (Nanog, Oct-4, and SOX2) are selected for further analysis. The selected cell populations are most likely to possess stem cell-like properties and are also the most promising for application in the regeneration of nucleus pulposus tissue to achieve therapeutic purposes, as these genes (Nanog, Oct-4, and SOX2) are typically associated with the maintenance and pluripotency of stem cells. In FIG. 2D, hollow circles indicate that the gene expression ratio has reached the selection threshold of greater than 1.5, while solid circles indicate that the expression ratio for all three genes has exceeded the selection threshold, with the cell populations that were finally selected and subjected to further analysis including NPP3, NPP7, and NPP19.

### Embodiment 3: Experiment of Migratory Property

In this embodiment, the cell populations NPP3, NPP7, and NPP19 screened based on embryonic stem cell genes further underwent a migratory property experiment to assess their migration ability. This experiment used Transwell culture plates to evaluate the migratory property of the cells. The transwell migration assay is a common method for measuring cell migration ability, wherein cells penetrate through a permeable membrane to the underside and adhere to the membrane, moving from one chamber to another, simulating the movement of cells in tissues.

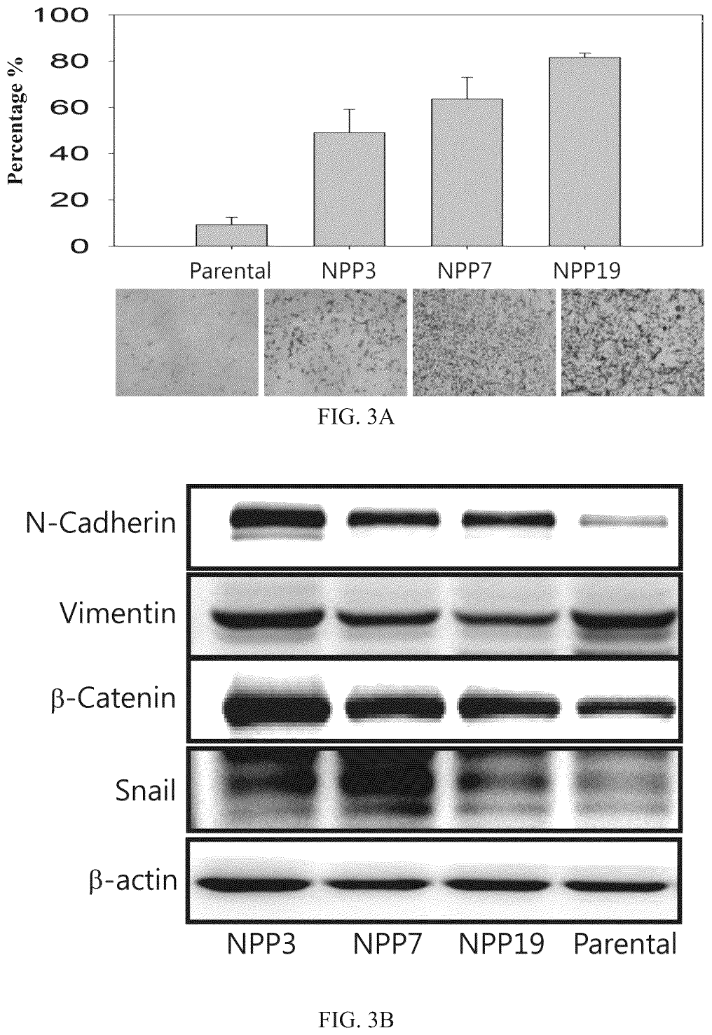

The bar chart in FIG. 3A shows the percentage of membrane penetration by the parental nucleus pulposus cell population (Parental) and the cell populations NPP3, NPP7, and NPP19. NPP19 showed the highest percentage of penetration, indicating NPP19 had the strongest migratory property in the experiment, followed by NPP7, while NPP3 displayed a lower migratory property. The migratory property of the parental nucleus pulposus cell population was significantly lower. The images below the bar chart are of cells stained with hematoxylin, which clearly shows the nuclei of the cells, allowing for the counting of cells that penetrated the membrane.

In the migratory property experiment of the embodiment, the expression of proteins related to cell migration was further analyzed using Western blotting. The proteins analyzed included N-Cadherin, Vimentin, β-Catenin, and Snail, which are essential for cell adhesion, cytoskeletal structure, and cell migration property. FIG. 3B shows that the cell populations NPP3, NPP7, and NPP19 all expressed these proteins. β-actin was used as an internal reference protein to ensure consistent protein loading amounts across the samples.

### Embodiment 4: Characterization of Mesenchymal Stem Cell (MSCs) Characteristics

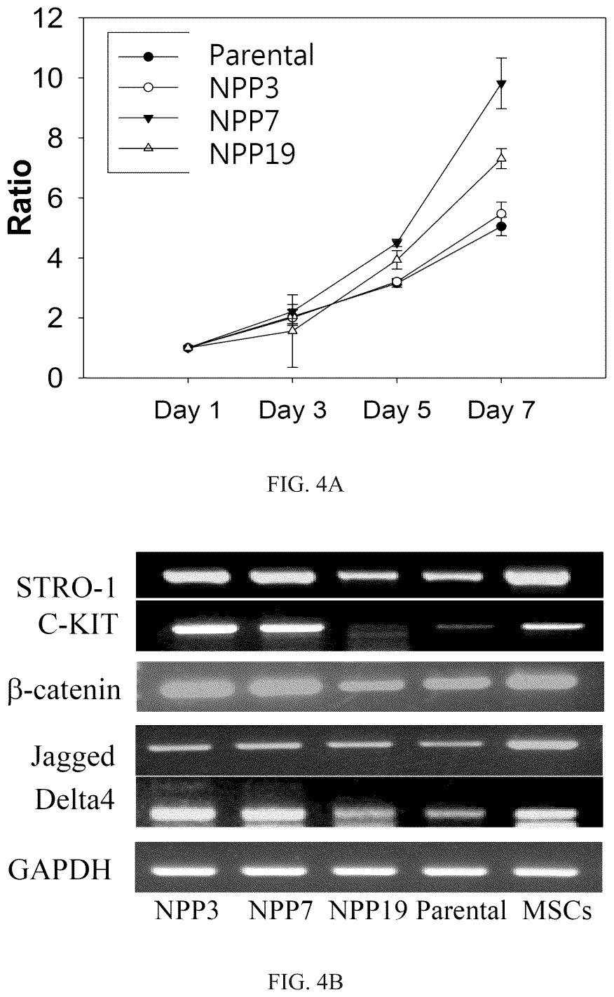

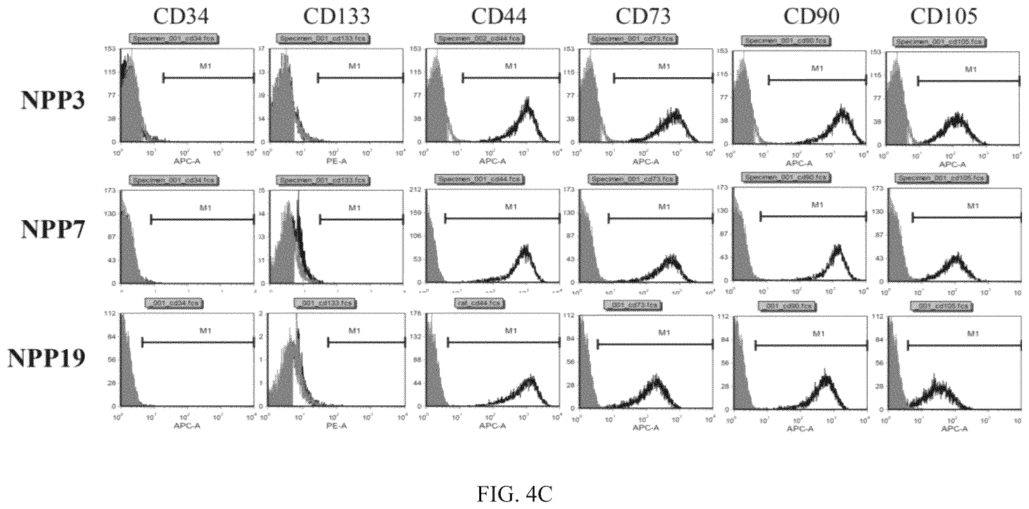

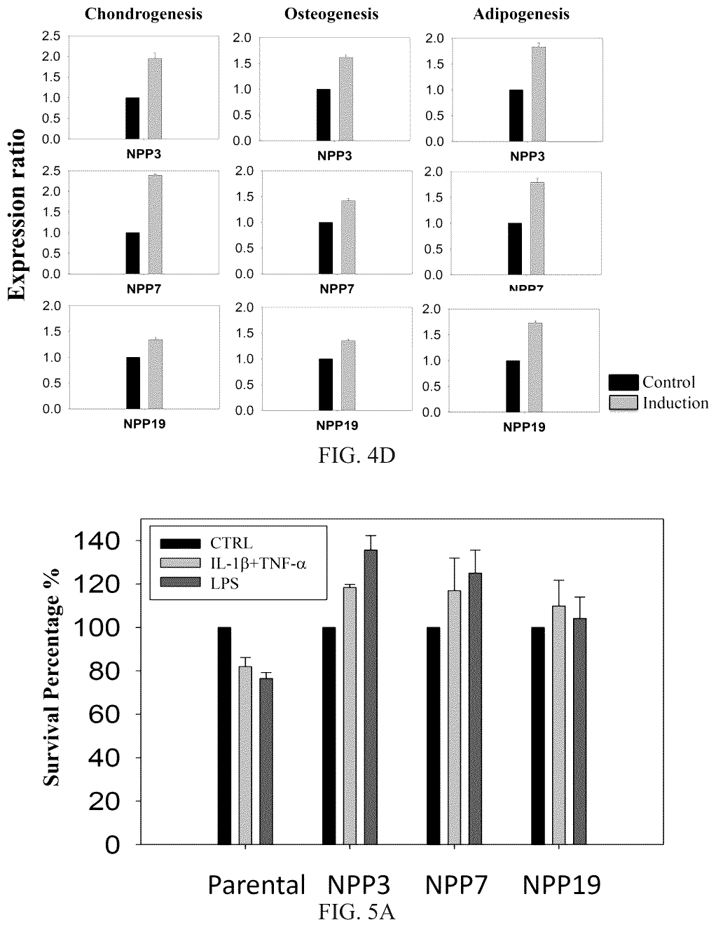

Referring to FIGS. 4A-4D, in the embodiment, the previously selected cell populations NPP3, NPP7, and NPP19 were again subjected to characterization experiments for mesenchymal stem cell characteristics. FIG. 4A shows the proliferation conditions of different cell populations (Parental and the cell populations NPP3, NPP7, and NPP19) on days 1, 3, 5, and 7 of culture. All cell populations proliferated over time, as indicated by the increase in ratios. On day 1, the proliferation ratio of all cell populations was close to 1, indicating that all cell populations started at approximately the same point at the beginning of the culture. Comparing the different time points, NPP7 showed a higher proliferation ratio than the other cell populations on days 5 and 7, especially on day 7 where the proliferation ratio of NPP7 is about 10, significantly higher than the other cell populations, indicating a stronger proliferation capability at these time points. The proliferation ratio of NPP19 fell between those of NPP7 and NPP3 on days 5 and 7. On day 5, the proliferation ratio of NPP3 was not different from that of the parental nucleus pulposus cell population; however, on day 7, the proliferation ratio of NPP3 was somewhat higher than that of the parental nucleus pulposus cell population. The higher proliferation ratios of NPP7 and NPP19 indicated stronger proliferation capabilities, allowing for a quicker supply of the required cell quantity, thereby more effectively repairing the damaged nucleus pulposus tissue and achieving the therapeutic goal of nucleus pulposus tissue regeneration.

FIG. 4B shows the expression of genes highly associated with mesenchymal stem cells (MSCs)—STRO-1, C-KIT, β-catenin, Jagged, and Delta4—used in characterizing the selected cell populations NPP3, NPP7, and NPP19, with the GAPDH gene serving as an internal reference gene (internal control). The three cell populations NPP3, NPP7, and NPP19 all expressed these five mesenchymal stem cell genes, indicating that they retained mesenchymal stem cell characteristics. Compared with the parental nucleus pulposus cell population, NPP3, NPP7, and NPP19 exhibited higher expression of the mesenchymal stem cell genes. The expression levels of all five genes in NPP3 and NPP7 were overall higher than those in NPP19, especially for STRO-1, C-KIT, and β-catenin, suggesting that NPP3 and NPP7 possessed more pronounced mesenchymal stem cell characteristics and may participate more effectively in the repair and regeneration of nucleus pulposus tissue.

FIG. 4C shows the analysis results of the expression of six different stem cell surface antigens (CD markers): CD34, CD133, CD44, CD73, CD90, and CD105, for the selected cell populations NPP3, NPP7, and NPP19 using a flow cytometer. NPP3, NPP7, and NPP19 expressed antigens CD44, 73, 90, and 105 on their surface, which are typical surface markers of mesenchymal stem cells. Therefore, it indicates that the characteristics of the cell populations NPP3, NPP7, and NPP19 are close to those of mesenchymal stem cells, potentially possessing similar functions and differentiation potential suitable for the repair and regeneration of nucleus pulposus tissue. Additionally, NPP3, NPP7, and NPP19 did not express the hematopoietic stem cell surface antigens such as CD34 and 133, further confirming their similarity to mesenchymal stem cells rather than hematopoietic stem cell characteristics.

FIG. 4D shows the gene expression ratios of the selected cell populations NPP3, NPP7, and NPP19 under three different differentiation conditions, with black bars representing the expression levels of the control group and gray bars representing the expression levels of the induction group. The cell populations NPP3, NPP7, and NPP19 were able to increase the gene expression ratios related to chondrogenesis, osteogenesis, and adipogenesis respectively, under the induction conditions for chondrogenesis, osteogenesis, and adipogenesis. Such multi-directional differentiation capability is significant for tissue engineering and regenerative medicine, as these cells can be used to repair or replace damaged nucleus pulposus tissue.

### Embodiment 5: Experiment of Anti-inflammatory Capability

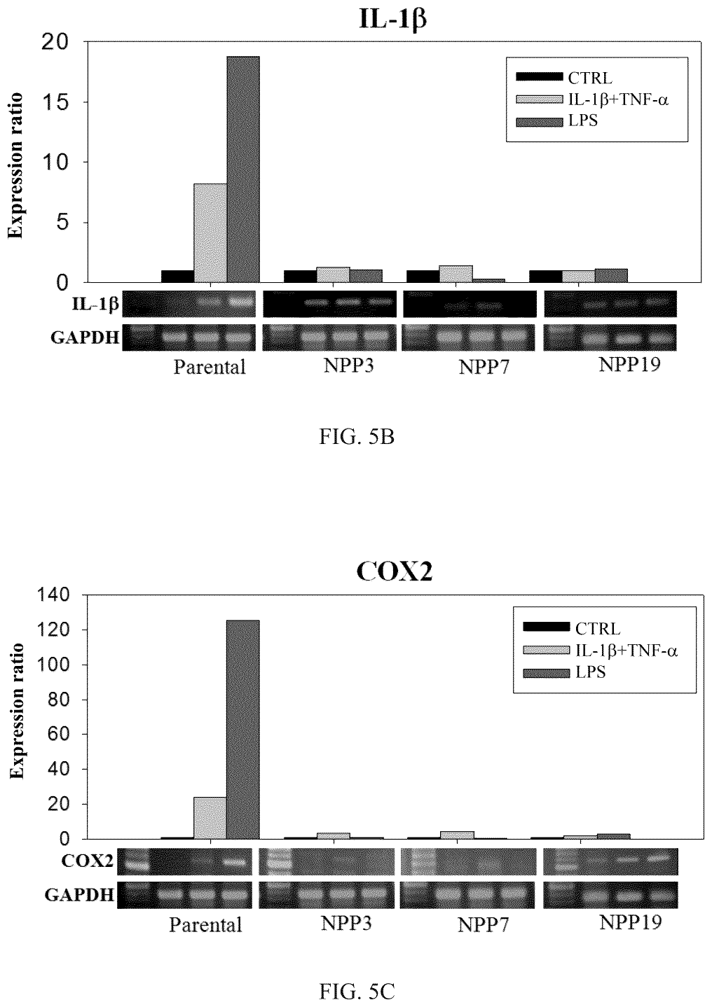

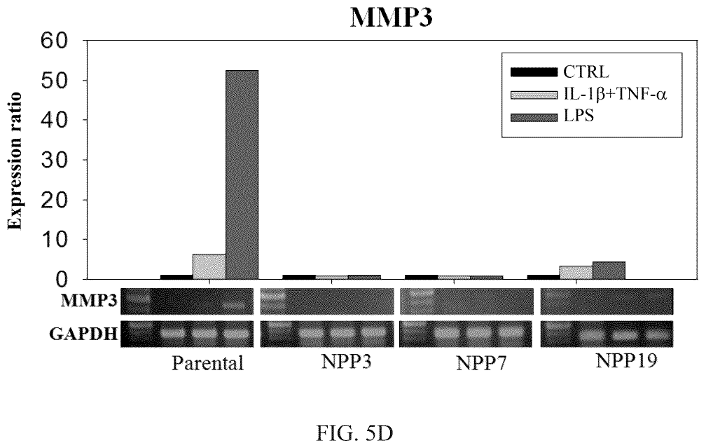

Referring to FIG. 5A-5D, in the embodiment, the previously selected cell populations NPP3, NPP7, and NPP19 were further characterized through anti-inflammatory capability experiments. The anti-inflammatory capability experiment includes identifying the survival percentage of the parental nucleus pulposus cell population and the cell populations NPP3, NPP7, and NPP19 under inflammatory environments (IL-1β+TNF-α and LPS (lipopolysaccharide) treatment); and characterizing the expression levels of inflammatory genes (IL-1β, COX-2, and MMP3) in the parental nucleus pulposus cell population and the cell populations NPP3, NPP7, and NPP19 under inflammatory environments.

FIG. 5A shows the survival percentage of the parental nucleus pulposus cell population and the cell populations NPP3, NPP7, and NPP19 under normal conditions (CTRL, control group) and inflammatory environments (treated with IL-1P+TNF-α and LPS). From FIG. 5A, it is evident that under inflammatory environments, the cell growth of the parental nucleus pulposus cell population was inhibited, thus reducing its survival rate, while the survival rates of the cell populations (NPP3, NPP7, NPP19) did not significantly decrease. This indicates that the cell populations (NPP3, NPP7, NPP19) have better resistance to the inflammatory environments, allowing them to continue growing and proliferating.

FIG. 5B shows the expression ratio of the inflammatory gene IL-1β in the parental nucleus pulposus cell population and the cell populations NPP3, NPP7, and NPP19 under normal conditions (CTRL, control group) and inflammatory environments (treated with IL-1β+TNF-α and LPS). The parental nucleus pulposus cell population exhibited a significant increase in IL-1β gene expression under inflammatory environments, whereas such an increase was not evident in the cell populations (NPP3, NPP7, NPP19), suggesting a certain inhibitory effect of these cell populations on the inflammatory response, making them less susceptible to disturbance.

FIG. 5C shows the expression ratio of the inflammatory gene COX-2 in the parental nucleus pulposus cell population and the cell populations NPP3, NPP7, and NPP19 under normal conditions (CTRL, control group) and inflammatory environments (treated with IL-1P+TNF-α and LPS). Similar to the IL-1βgene, the expression of COX-2 increased in the parental nucleus pulposus cell population under inflammatory environments, while such an increase was not evident in the cell populations (NPP3, NPP7, NPP19), further supporting the anti-inflammatory properties of these cell populations.

FIG. 5D shows the expression ratio of the inflammatory gene MMP3 in the parental nucleus pulposus cell population and the cell populations NPP3, NPP7, and NPP19 under normal conditions (CTRL, control group) and inflammatory environments (treated with IL-1β+TNF-α and LPS). Consistent with the trends observed for the previous two inflammatory genes (IL-1β and COX-2), the expression ratio of MMP3 significantly increased in the parental nucleus pulposus cell population under inflammatory environments, while the expression levels were minimally affected in the cell populations (NPP3, NPP7, NPP19).

From the aforementioned anti-inflammatory capability experiments, it is evident that the nucleus pulposus progenitor cells selected by the present invention (the cell populations NPP3, NPP7, and NPP19) show a stronger survival ability and lower expression of inflammatory genes under inflammatory environments compared with the parental nucleus pulposus cell population. This indicates that the cell populations NPP3, NPP7, and NPP19 possess a higher anti-inflammatory potential, which is very valuable in clinical applications, especially in situations where controlling the inflammatory response is necessary, such as in the repair and treatment of nucleus pulposus tissue.

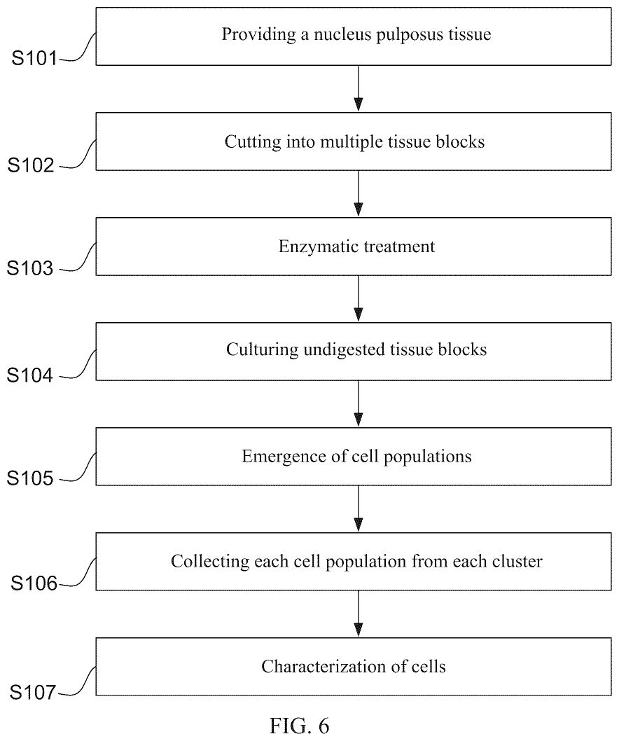

FIG. 6 is a flowchart of the method for isolating and characterizing nucleus pulposus progenitor cells according to the present invention, including the following steps:
S101 (Providing a nucleus pulposus tissue): surgically removing the nucleus pulposus tissue specimen from patients with intervertebral disc degeneration or disc herniation in a sterile manner;
S102 (Cutting into multiple tissue blocks): then cutting the nucleus pulposus tissue specimen into a plurality of tissue blocks;
S103 (Enzymatic treatment): hydrolyzing the tissue blocks with an enzyme, such as collagenase or trypsin;
S104 (Culturing undigested tissue blocks): washing with DPBS to remove the enzyme, then culturing the undigested tissue blocks in a culture dish for 1 to 7 days;
S105 (Emergence of cell populations): after culturing the undigested tissue blocks in a culture dish for 1 to 7 days, a plurality of cell populations emerge (crawl out) from the tissue blocks and form a plurality of clusters at the bottom of the culture dish;
S106 (Collecting each cell population from each cluster): collecting each cell population from each cluster and separately culturing each of them in different culture dishes for expansion; and
S107 (Characterization of cells): then, using the method described in Embodiment 2 above, screening for nucleus pulposus progenitor cells from each cell population using the embryonic stem cell genes Nanog, Oct-4, and SOX2, followed by further characterizing the migratory property, mesenchymal stem cell (MSC) characteristics, and anti-inflammatory capability of the selected nucleus pulposus progenitor cells using the methods shown in Embodiments 3 to 5, to obtain the nucleus pulposus progenitor cells most suitable for nucleus pulposus tissue regeneration.

The above descriptions have comprehensively introduced the method for isolating and characterizing nucleus pulposus progenitor cells and for improving lower back pain using the nucleus pulposus progenitor cells obtained according to the present invention. It should be emphasized that the above descriptions are made on embodiments of the present invention; however, the embodiments are not intended to limit the scope of the present invention, and all equivalent implementations or alterations within the spirit of the present invention still fall within the scope of the present invention.

What is claimed is:

1. A method for isolating and characterizing nucleus pulposus progenitor cells, comprising:
a) providing a nucleus pulposus tissue, and cutting the nucleus pulposus tissue into a plurality of tissue blocks;
b) hydrolyzing the tissue blocks with an enzyme;
c) removing the enzyme and culturing the tissue blocks in a culture dish; and
d) when a plurality of cell populations emerge from the tissue blocks and form a plurality of clusters at the bottom of the culture dish, screening for the nucleus pulposus progenitor cells within each of the cell populations in each cluster, using an embryonic stem cell gene, wherein the embryonic stem cell gene is selected from the group consisting of Nanog, Oct-4, and SOX2.

2. The method of claim 1, wherein the nucleus pulposus progenitor cells with migratory property can be further screened using at least one protein selected from the group consisting of N-Cadherin, Vimentin, β-Catenin, and Snail protein.

3. The method of claim 1, wherein the nucleus pulposus progenitor cells with mesenchymal stem cell characteristics can be further screened using at least one gene selected from the group consisting of STRO-1, C-KIT, β-catenin, Jagged, and Delta4.

4. The method of claim 1, wherein the nucleus pulposus progenitor cells with mesenchymal stem cell characteristics can be further screened using at least one stem cell surface antigen selected from the group consisting of CD34, CD44, CD73, CD90, CD105, and CD133.

5. The method of claim 1, wherein the nucleus pulposus progenitor cells with a differentiation capability can be further screened using the differentiation capability selected from the group consisting of chondrogenesis, osteogenesis, and adipogenesis.

6. The method of claim 1, wherein the nucleus pulposus progenitor cells with an anti-inflammatory capability can be further screened using at least one inflammation-related gene selected from the group consisting of IL-1β, COX-2, and MMP3.

7. The method of claim 1, wherein the enzyme for hydrolyzing the tissue blocks is collagenase, trypsin, or a combination thereof.

8. A method for improving lower back pain comprising: administering an effective amount of a pharmaceutical composition containing the nucleus pulposus progenitor cells obtained by the method of claim 1 to a subject suffering from lower back pain.

9. The method of claim 8, wherein the nucleus pulposus progenitor cells with migratory property can be further screened using at least one protein selected from the group consisting of N-Cadherin, Vimentin, β-Catenin, and Snail protein, and/or the nucleus pulposus progenitor cells with mesenchymal stem cell characteristics can be further screened using at least one stem cell surface antigen selected from the group consisting of CD34, CD44, CD73, CD90, CD105, and CD133.

10. The method of claim 9, wherein the nucleus pulposus progenitor cells with a differentiation capability can be further screened using the differentiation capability selected from the group consisting of chondrogenesis, osteogenesis, and adipogenesis; and/or wherein the nucleus pulposus progenitor cells with an anti-inflammatory capability can be further screened using at least one inflammation-related gene selected from the group consisting of IL-1β, COX-2, and MMP3.
